(19) United States
(12) Patent Application Publication
(10) Pub. No.: US 2006/0228756 A1
(43) Pub. Date: Oct. 12, 2006

(54) Relational database management system for automated random crystallization screening

(75) Inventors: Brent W. Segelke, San Ramon, CA (US); April A. Newman, Livermore, CA (US); Heike I. Krupka, Livermore, CA (US); Timothy P. Lekin, Livermore, CA (US)

Correspondence Address: Lawrence Livermore National Laboratory;Assistant Laboratory Counsel, L-703, P.O. Box 808, Livermore, CA, 94551, US

(73) Assignee: The Regents of the University of California

(21) Appl. No.: 11/353,492

(22) Filed: Feb. 13, 2006

Related U.S. Application Data
(60) Provisional application No. 60/652,476, filed on Feb. 11, 2005.

(51) Int. Cl. G06F 19/00; G01N 33/53

(52) U.S. Cl. 435/7.1; 702/19

(57) ABSTRACT

A relational database management system for automated random crystallization screening systems so as to provide facilitated data tracking, maintenance, and analysis. The system includes a database server module capable of storing data; an ARCS module having a crystallization screen design engine capable of generating random crystallization screens and associated crystallization experiments, and a data entry and query applications module capable of passing data between the database server module and a user. The database server module operates to correlate the data received from the ARCS module and the data entry and query applications module with sample data, to organize the data so as to systematically reveal, for example, conditions that do and do not lead to crystal growth.

### I. CLAIM OF PRIORITY IN PROVISIONAL APPLICATION

This application claims the benefit of U.S. provisional application No. 60/652,476 filed Feb. 11, 2005, entitled, “Database for Data Tracking and Analysis of Automated Random Crystallization Screening” by Brent W. Segelke et al.

The United States Government has rights in this invention pursuant to Contract No. W-7405-ENG-48 between the United States Department of Energy and the University of California for the operation of Lawrence Livermore National Laboratory.

### II. FIELD OF THE INVENTION

The present invention is related to protein crystallography, and is more particularly related to a relational database management system for data tracking and analysis of automated random crystallization screening.

### III. BACKGROUND OF THE INVENTION

Proteomics is the study of the structure of proteins and their function in an organism. Research efforts in this field have focused on obtaining atomic-resolution 3-D protein structures of whole genomes, such as by macromolecular/protein crystallography, which will ultimately provide representative structures for all individual protein families. One of the major bottlenecks, however, of protein crystallography and structural genomics has been and continues to be the limited availability of diffraction-quality protein crystals. Despite advances in rapid structure determination and automation of crystallization setups for high throughput, improvements in applied crystallization strategies (“screening strategies” or “screens”) which enable large-scale production of diffraction-quality protein crystals, have been limited.

There is a theoretically infinite spectrum (and practically, more than 30 million) of possible crystallization conditions (i.e. a combination of factors/parameters such as temperature, pH, ionic strength, specific concentration of precipitants and additives, etc.) affecting macromolecular solubility that can potentially lead to protein crystallization. State of the art protein crystallography techniques require empirical screening from this vast set of possible combinations to discover conditions that initiate de novo protein crystallization. Considering the usually limited amount of available protein, and the inconvenience, time factor, and expense of testing large numbers of combinations, setting up a complete set of crystallization trials is considered unrealistic. Consequently, conventional screening efforts are typically limited to a small finite set of pre-made conditions, i.e. pre-made screens, often based on a collection of crystallization recipes that have proven in the past to successfully produce crystals of at least one protein or slight variations thereof. However, dependence on such pre-made screens can limit the potential for successful crystallization screening experiments, as well as what might be learned about crystallization and the conditions leading to crystal growth.

U.S. Pat. No. 6,860,940, entitled “Automated Macromolecular Crystallization Screening” to Applicant, discloses one particular screening approach designed to automatically generate screens of crystallization conditions using a random search model, i.e. an automated random crystallization screening (ARCS) technique. Random screening was determined by Applicants in experiments performed for the Lawrence Livermore National Laboratory, to be the most effective way to assess the number of successful experiments in a given crystallization condition space without exhaustively covering its entire spectrum, and therefore to have the greatest average efficiency compared with conventional strategies. Furthermore, random screening requires fewer experiments to arrive at the first successful crystallization. By performing random sampling in the screening process, the '940 patent approaches protein crystal screening as a stochastic sampling problem. As such, this approach to crystallization screening enables the parameters effecting crystallization to be analyzed statistically as independent variables. Any number of random combinations of crystallization conditions may be generated from a large set of starting stock-solutions, and may be interfaced to an automated liquid-handling system, such as for example a commercially available Packard MPII. With current implementation, it is possible to setup up about 4000 experiments per day.

Automated screening capabilities, such as described in the '940 patent, create an additional challenge for data tracking and analysis. What is needed therefore is a system for supporting such ARCS systems to provide facilitated data tracking, maintenance, and analysis and which could be easily data-mined to learn more about crystallization, including conditions that do and do not lead to crystal growth.

### IV. SUMMARY OF THE INVENTION

One aspect of the present invention includes a computerized relational database management system (RDMS) for data tracking of automated random crystallization screening (ARCS), comprising: a database server module capable of storing data; an ARCS module having a crystallization screen design engine capable of generating a first set of random crystallization screens and associated crystallization experiments and subsequent sets of crystallization screens and crystallization experiments based on a preceding set, said ARCS module operably connected to the database server module to communicate crystallization screen data and crystallization experiment data therebetween; a data entry and query applications module operably connected to the database server module and capable of passing data between the database server module and a user, wherein the database server module correlates the data received from the ARCS module and the data entry and query applications module with sample data.

Another aspect of the present invention includes a method in a relational database management system for data tracking and analysis of automated random crystallization screening (ARCS), comprising: in a database server module capable of storing data, recording sample information received from a user via a data entry and query applications module operably connected to the database server module and capable of passing data between the database server module and the user; in the database server module, recording crystallization screen data designed by an ARCS module having a crystallization screen design engine capable of generating a first set of random crystallization screens and associated crystallization experiments and subsequent sets of crystallization screens and crystallization experiments based on a preceding set, said ARCS module operably connected to the database server module to communicate crystallization screen data and crystallization experiment data therebetween; in the database server module, correlating recorded data received from the ARCS module and the data entry and query applications module with sample data.

Another aspect of the present invention includes a memory for storing data for access by an application program being executed on a data processing system, comprising: a data structure stored in said memory, said data structure including information resident in a database used by said application program and including at least the following fields: a protein sample ID field; at least one protein sample attribute field(s) associated with each protein sample ID field; a plurality of crystallization screen ID fields associated with each sample ID; at least one reagent field(s) associated with each crystallization screen ID field; and a plurality of crystallization experiment ID fields associated with each crystallization screen ID.

Another aspect of the present invention includes a data processing system executing an application program and containing a database used by said application program, said data processing system comprising: CPU means for processing said application program; and memory means for holding a data structure for access by said application program, said data structure being composed of information resident in said database used by said application program and including at least the following fields: a protein sample ID field; at least one protein sample attribute field(s) associated with each protein sample ID field; a plurality of crystallization screen ID fields associated with each sample ID; at least one reagent field(s) associated with each crystallization screen ID field; and a plurality of crystallization experiment ID fields associated with each crystallization screen ID.

Another aspect of the present invention includes a computer readable medium containing a data structure for tracking data of an automated random crystallization system (ARCS), the data structure comprising: a protein sample ID field; at least one protein sample attribute field(s) associated with each protein sample ID field; a plurality of crystallization screen ID fields associated with each sample ID; at least one reagent field(s) associated with each crystallization screen ID field; and a plurality of crystallization experiment ID fields associated with each crystallization screen ID.

### V. BRIEF DESCRIPTION OF THE DRAWINGS

The accompanying drawings, which are incorporated into and form a part of the disclosure, are as follows:

FIG. 1 is a flow chart of an exemplary automated macromolecular crystallization screening system disclosed in U.S. Pat. No. 6,860,940.

FIG. 2 is a schematic block diagram of an embodiment of the present invention.

FIG. 3 is a schematic block diagram of an embodiment of the present invention illustrating data flow between modules.

FIG. 4 is a flow chart of an embodiment of the RDMS of the present invention, as it relates to the processing of a sample material shown running in parallel.

### VI. DETAILED DESCRIPTION

The present invention is directed to a relational database management system, “RDMS” for use with automated random crystallization screening (“ARCS”) systems and techniques, such as for example disclosed in U.S. Pat. No. 6,860,940 (hereinafter “'940 patent”) incorporated by reference herein in its entirety, to provide data tracking and analysis support to the computer-based crystallization screen design and setup of such systems. It is appreciated that a relational database is a database based on the relational model where data and relations between them are organized in tables comprising rows and fields. A relational database allows the definition of data structures, storage and retrieval operations and integrity constraints, as known in the art. Structured Query Language (SQL), an industry-standard language often embedded in general purpose programming languages, is preferably used for creating, updating and, querying the relational database.

A. Automated Random Crystallization Screening (ARCS)

In an ARCS process, such as described in the preferred example of the '940 patent, an initial set of screens produced from a random selection of premixed stock reagents is used in a first round of crystallization experiments, with subsequent screens and crystallization experiments designed and performed based on the results of the preceding round in automated fashion. A general description of the ARCS process follows. Preferably, screen design software/computer (random crystallization design engine) is integrated with a liquid handling robot which is programmed to handle the run time instructions supplied by the design software, in order to mix crystallization cocktails (i.e. screens) from stock reagents. A multiplicity of crystallization experiments are then set up on analysis plates by combining protein samples to the prepared screens. A second robot may also be used to set up the crystallization experiments by transferring the prepared screens to crystallization plates and combining protein samples to the screens. Instructions for the second robot are also provided by the design software/computer. The analysis plates are then incubated to promote growth of crystals in the analysis plates. The crystallization experiments observed at regular intervals, such as with a CCD microscope camera (for crystal imaging), and observations are scored to determine crystal formation. The images are analyzed with regard to expected suitability of the crystals for analysis by x-ray crystallography. If the crystals are not ideal, a second set of screens are designed (not random) by the screen design software, produced, and used in a second round of crystallization experiments of the sample. Additional rounds of screen designs and crystallization experiments may be performed in a similar fashion depending on the expected suitability for x-ray crystallography, with each subsequent screen design based on crystallization results of the previous round.

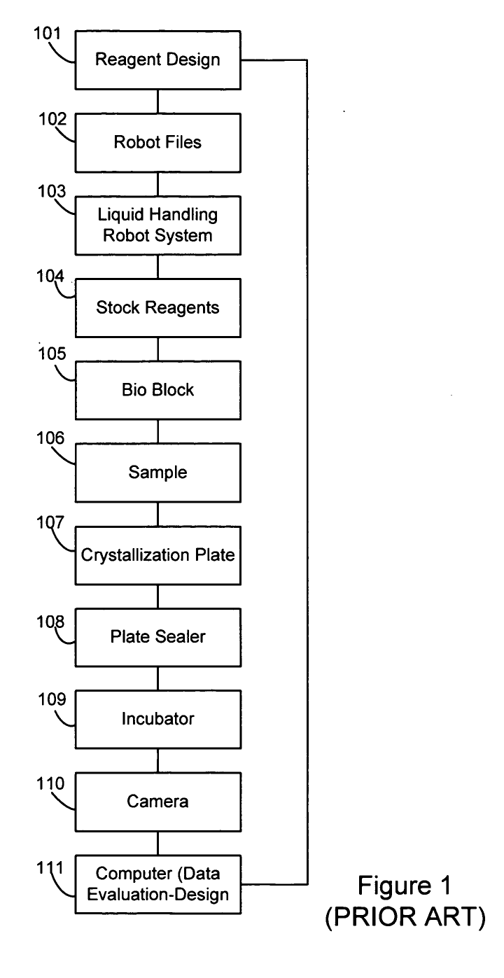

FIG. 1 shows a flow diagram illustrating a particular ARCS process described in the '940 patent as follows. A reagent design 101 is used to create a set of robot files 102. The reagent design is used by a liquid handling robot system 103 to randomly select reagent components from a set of stock reagents 104 and create a multiplicity of reagent mixes in bioblock 105. The initial reagent design is a purely random reagent design. Sample 106 and bioblock 105 are used with a crystallization plate 107 to create a multiplicity of individual analysis plates within crystallization plate 107 wherein each of the analysis plates receives a set format of the reagent mixes combined with the sample. The crystallization plate 107 is sealed by plate sealer 108 and transferred to an incubator 109 for incubation. Incubation promotes growth of crystals in the analysis plates. A camera 110 is used to create images of the crystals in the analysis plates. A computer 111 analyzes the images with regard to suitability of the crystals for analysis by x-ray crystallography. The computer 111 provides a reagent mix design that produces specific reagent mixes that are expected to produce the best crystals for analysis by x-ray crystallography. The reagent mix design is used to create a second multiplicity of mixes of the reagent components. The second multiplicity of reagent mixes are used for another round of automated macromolecular crystallization screening the sample. The second round of automated macromolecular crystallization screening may produce crystals that are suitable for x-ray crystallography. If the second round of crystallization screening does not produce crystals suitable for x-ray crystallography a third reagent mix design is created and analyzed according to the method.

B. RDMS Operation

Generally, the RDMS of the present invention is an integrated computer-based platform for tracking information related to a received protein sample, as well as crystallization screen conditions/setup and experiment results data produced by an ARCS process (as described above), and making the results and related data available for analysis. The routine processing of samples for crystallization requires the tracking of, for example: samples received, properties and history of samples received, aliquots made from samples received, chemicals for crystallization screening, reagents made from chemicals, screens made from crystallization reagents, experiments setup by combination of screens with samples received, observations (digital images produced by the robotic CCD camera), results from observations, etc. By enabling the tracking of these and other aspects associated with a protein sample, the database of crystallization experiments provides new opportunities to study the correlations between individual parameters and crystallization results as well as combinations of parameters and their effects on crystallization, in order to enable more rigorous and fundamental studies to be made about crystallization screening itself.

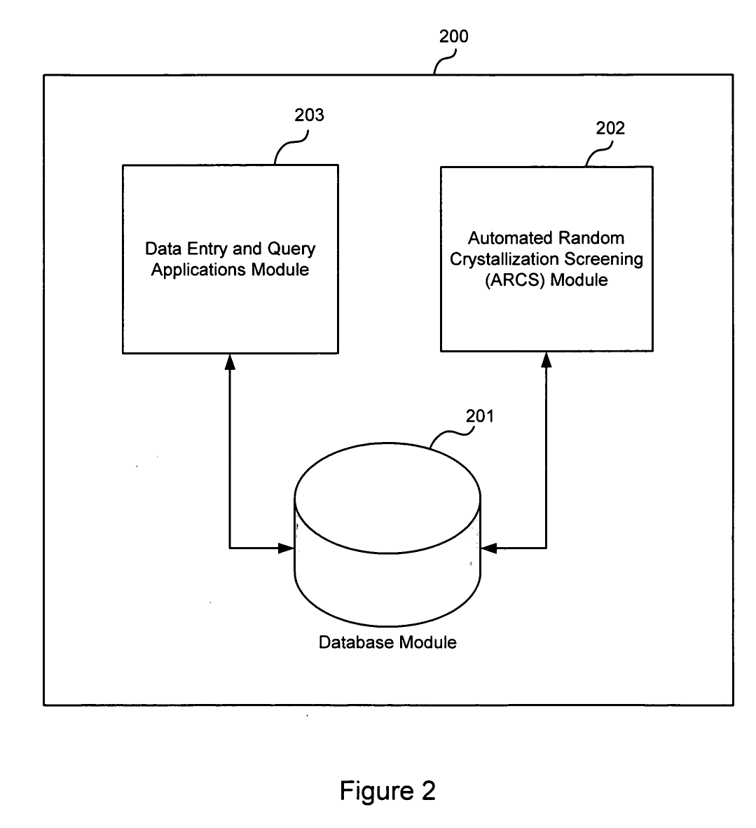

The RDMS of the present invention may be generally characterized as comprising various data collection applications, a database server, and data stored on the database server. As such the RDMS 200 is shown in FIG. 2 as having three top-level modules, including a database server module 201 for data storage and access, an ARCS system module 202 including a crystallization design engine for generating screen setup/crystallization experiment data, and a data entry/query applications module 203 for enabling data entry by users and making data available to users. The data server module 201 is operably connected to both the ARCS system module 202 and the data entry/query applications module 203 to pass data therebetween. Sample information from the data entry module 203, and screen setup conditions and results from the design engine module 202 are recorded/archived in (preferably automatically) and accessed from the database module 201, as indicated by arrows. And in the database server module 201, the screen and crystallization experiment data are linked, associated, or otherwise correlated to a particular sample (aliquot) to enable tracking thereof. As discussed in Section A, the ARCS system module 202 may also include instrument integration by which screen setup and crystallization experiments are implemented by robots via robot instructions.

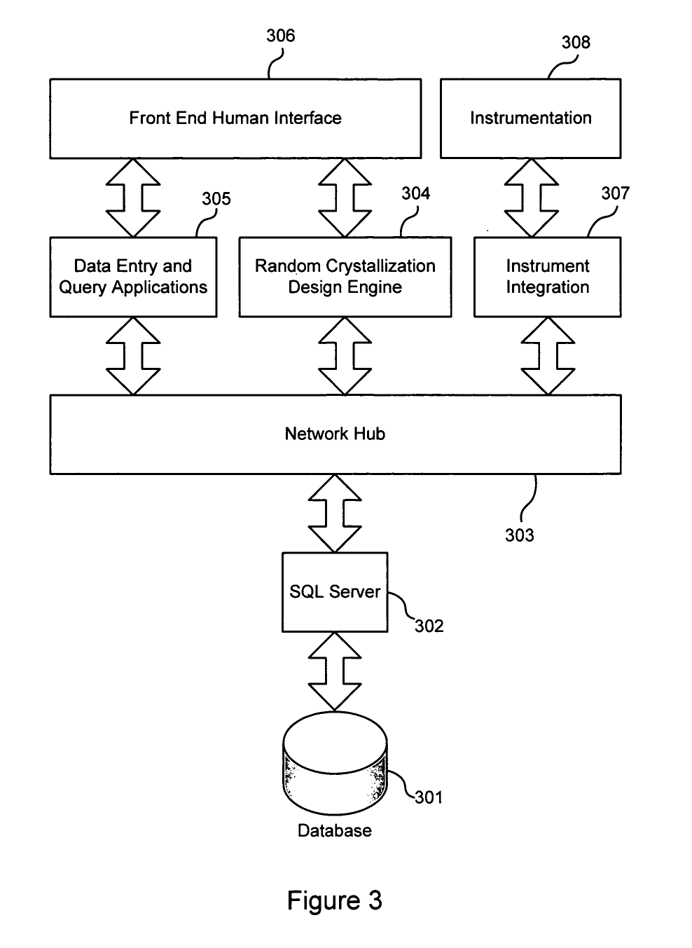

FIG. 3 shows a schematic block diagram of a preferred embodiment of the RDMS of the present invention, illustrating exemplary data flow between component modules, and in particular to/from a database shown at block 21 via a SQL server 302. The top row in FIG. 3 shows that data may originate from or be delivered to either a human user via a human interface 306, or an instrument 308 such as the robots/machines for implementing the reagent mixing described in the '940 patent. And the second row in FIG. 3 shows three data processing modalities/applications by which data storage and retrieval from the database 301 is implemented, including a data entry and query applications module 305, a random crystallization design engine module 304 (part of an ARCS system), and an instrument integration module 307 (which may also be part of the ARCS system as previously described). The third row in FIG. 3 shows a network hub 303 of a type known in the art by which the multiple applications connect to and communicate with the SQL server 302 and the database 301.

The random crystallization design engine module 304 of the ARCS system serves to create screen designs, crystallization experiments, and robot instructions to carry out those experiments, as previously described in part A. These types of data are preferably automatically archived in the database, and correlated to a sample. Robot instructions may be sent directly to the instruments 308 via the network hub 303 and instrument integration 307 to carry out specified tasks, such as part of the ARCS system. And data results from the instruments (e.g. CCD camera) may be entered into the database for observation and analysis.

The data entry and query applications module 305 enables users to directly enter/retrieve data from the database 301. For example, a web-based form may be used to provide sample information when a user first announces his intention to supply the sample material. Web forms may also be provided to allow for specific queries of the database, such as to query information related to received samples, received chemicals, stock reagents, labware for crystallization experiments, results, etc., as well as crystallization condition information for an observed crystal. Preferably, sample materials and setup configurations are tracked with barcodes provided by the RDMS in the database 301 to facilitate tracking as data is passed between modules.

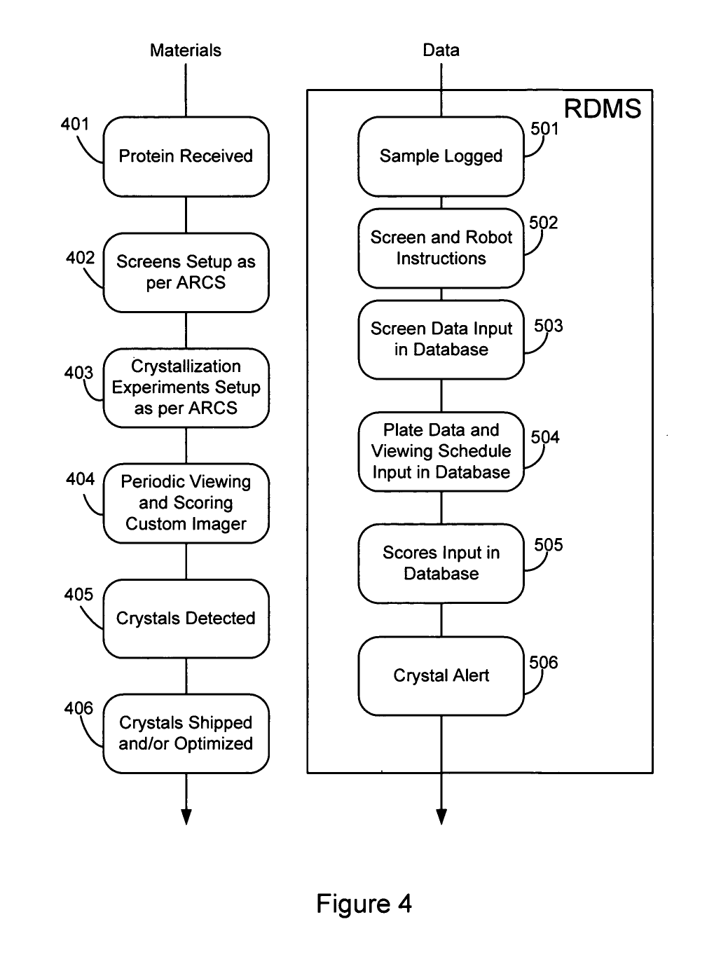

FIG. 4 shows a comparison of the processing/tracking of materials in an ARCS system (left column), and the associated data flow (right column) running in parallel. First, sample protein is received at a crystallization facility, as indicated at block 401, and the sample is logged into the RDMS at block 501. It is appreciated that sample logging at 501 may include data entry by a user prior to submitting the sample, indicating his intention to submit the sample for crystallization experiments, and providing sample information. This may be accomplished via a web form interface. After receiving the sample, the sample may be further catalogued in the database, such as via a second web form interface. In any case, various attributes of the sample materials can be catalogued including, for example: purity information, size, composition, buffer conditions, concentration, chain of custody, etc. It is notable that after a sample is received, it may be divided into aliquots depending on the quantity of sample received. Therefore, sample logging may further include cataloguing each aliquot, and labeling each aliquot with a barcode to facilitate tracking.

At this point, the crystallization screen design software of the ARCS system is executed to produce recipes for novel crystallization screens. In particular, a first random screen design (reagent mixture specifications) is prepared by the ARCS system (not shown) via the random crystallization design engine, including robot instructions for carrying out the crystallization experiments. As shown at block 502, these screen and robot instructions are inputted into the database for the corresponding aliquot. Once recorded, the new screens are set up as per ARCS (e.g. via integrated instruments) at block 402 and the corresponding screen data is input in the database at block 503. It is appreciated that an application may be provided residing on the computer and interfaced with the liquid handling robot to act as a plug-in to interpret output from the crystallization design software. This plug-in application is preferably configured to populate the database with the information about the crystallization screen sufficient to fully reconstruct each screen. Also, a barcode may be generated to label each new screen, so as to facilitate screen identification by scanning the barcode.

At block 403, the crystallization experiments are next set up by combining the sample with the various screens on a crystallization plate, as per ARCS, and the corresponding plate data and viewing schedule is input in the database at block 504. Crystallization plates are preferably cataloged via a web form where the barcode for the sample aliquot and the barcode for the screen are similarly entered. Preferably, another barcode is generated by the RDMS to identify the newly set-up crystallization plates. Block 504 also shows that the RDMS generates a viewing schedule for each plate. And the RDMS keeps a list of e-mail addresses for researchers that are responsible for the viewing of crystallization experiments.

At block 404, the crystallization plates are periodically viewed, as per the viewing schedule, and scored, such as by using an imager and automatic crystal detection software. In particular, the crystallization plates may be regularly scanned by a CCD microscope camera that is equipped with a bar code scanner for identifying the particular aliquot, screen, and crystallization experiment. And at block 505, the CCD images and scores of crystallization experiments are input into the database. Preferably, an application running on the computer which controls the CCD microscope camera operates to populate the database with http links to images acquired from crystallization experiments and scores produced by the crystal detection software. A web form may additionally be provided to allow for the manual entry of scores into the database by researchers.

Upon detection of crystals at block 405, an alert is issued by the RDMS at 506. Preferably, an e-mail is sent to designated confirmers for confirmation of crystallization when a new crystal is reported and to allow for immediate processing of newly discovered crystals. Additionally, one particular function which may be provided by the data entry and query applications module 305 of FIG. 3 is a report generating function providing a summary of crystallization experiments. For example, regular reports may be provided on, for example: the number and identification of samples in process, the number of screens produced, the number of experiments performed, the mean, minimum, and maximum score for each sample, and the percentage of experiments that lead to crystallization for each sample.

And at step 406, detected crystals may be shipped and/or optimized. In total, the database relieves the substantial work load of data tracking and archiving and allows for rapid reporting of results and conditions that lead to crystallization.

The RDMS present invention may be used, for example, for applications involving structural genomics, high-throughput x-ray crystallography, proteomics, biomedical research, basic biology research, public health, biodefense. Other applications may involve high-throughput macromolecular structure determination by x-ray crystallography, proteomics, drug design, and pharmaceutical research.

While particular operational sequences, materials, temperatures, parameters, and particular embodiments have been described and or illustrated, such are not intended to be limiting. Modifications and changes may become apparent to those skilled in the art, and it is intended that the invention be limited only by the scope of the appended claims.

I claim:

1. A computerized relational database management system (RDMS) for data tracking of automated random crystallization screening (ARCS), comprising:
a database server module capable of storing data;
an ARCS module having a crystallization screen design engine capable of generating a first set of random crystallization screens and associated crystallization experiments and subsequent sets of crystallization screens and crystallization experiments based on a preceding set, said ARCS module operably connected to the database server module to communicate crystallization screen data and crystallization experiment data therebetween;
a data entry and query applications module operably connected to the database server module and capable of passing data between the database server module and a user,
wherein the database server module correlates the data received from the ARCS module and the data entry and query applications module with sample data.

2. The RDMS of claim 1,
wherein the ARCS module automatically archives crystallization screen data and crystallization experiment data to the database server module upon generation thereof.

3. The RDMS of claim 1,
wherein the ARCS module generates barcodes for the crystallization screens and barcodes for the associated crystallization experiments upon generation thereof.

4. The RDMS of claim 1,
wherein the ARCS module includes an instrument integration module for implementing the crystallization screens and associated crystallization experiments via operably connected crystallization instruments.

5. The RDMS of claim 4,
wherein the instrument-integration module includes an imaging system capable of imaging the crystallization experiments and archiving the images in the database server module.

6. The RDMS of claim 5,
wherein the instrument integration module includes crystal detection means for detecting crystals from said images and archiving detection scores to the database server module.

7. The RDMS of claim 1,
wherein the data entry and query applications module generates barcodes for sample aliquots entered into the RDMS for tracking thereof.

8. The RDMS of claim 1,
wherein the data entry and query applications module includes a network-based data entry form for recording in the database server module sample information from a user.

9. The RDMS of claim 1,
wherein the data entry and query applications module includes a network-based entry form for recording in the database server module detection scores from a reviewer.

10. The RDMS of claim 1,
wherein the data entry and query applications module includes a report generator.

11. A method in a relational database management system for data tracking and analysis of automated random crystallization screening (ARCS), comprising:
in a database server module capable of storing data, recording sample information received from a user via a data entry and query applications module operably connected to the database server module and capable of passing data between the database server module and the user;
in the database server module, recording crystallization screen data designed by an ARCS module having a crystallization screen design engine capable of generating a first set of random crystallization screens and associated crystallization experiments and subsequent sets of crystallization screens and crystallization experiments based on a preceding set, said ARCS module operably connected to the database server module to communicate crystallization screen data and crystallization experiment data therebetween;
in the database server module, correlating recorded data received from the ARCS module and the data entry and query applications module with sample data.

12. The method of claim 11,
further comprising automatically recording crystallization screen data and crystallization experiment data to the database server module upon generation thereof.

13. The method of claim 11,
further comprising generating barcodes for the crystallization screens and barcodes for the associated crystallization experiments upon generation thereof.

14. The method of claim 11,
further comprising recording in the database server module images of the crystallization experiments imaged by an imaging system of an instrument integration module of the ARCS module.

15. The method of claim 14,
further comprising recording in the database server module detection scores generated by crystal detection means of the instrument integration module of the ARCS module.

16. The method of claim 11,
further comprising generating barcodes for sample aliquots entered into the RDMS via the data entry and query applications module, for tracking thereof.

17. A memory for storing data for access by an application program being executed on a data processing system, comprising:
a data structure stored in said memory, said data structure including information resident in a database used by said application program and including at least the following fields:
a protein sample ID field;
at least one protein sample attribute field(s) associated with each protein sample ID field;
a plurality of crystallization screen ID fields associated with each sample ID;
at least one reagent field(s) associated with each crystallization screen ID field; and
a plurality of crystallization experiment ID fields associated with each crystallization screen ID.

18. The memory of claim 17,
wherein the sample ID field is a barcode ID field.

19. The memory of claim 17,
wherein the plurality of crystallization screen ID fields are barcode ID fields

20. The memory of claim 17,
wherein the plurality of crystallization experiment ID fields are barcode ID fields.

21. A data processing system executing an application program and containing a database used by said application program, said data processing system comprising:
CPU means for processing said application program; and
memory means for holding a data structure for access by said application program, said data structure being composed of information resident in said database used by said application program and including at least the following fields:
a protein sample ID field;
at least one protein sample attribute field(s) associated with each protein sample ID field;
a plurality of crystallization screen ID fields associated with each sample ID;
at least one reagent field(s) associated with each crystallization screen ID field; and
a plurality of crystallization experiment ID fields associated with each crystallization screen ID.

22. A computer readable medium containing a data structure for tracking data of an automated random crystallization system (ARCS), the data structure comprising:
a protein sample ID field;
at least one protein sample attribute field(s) associated with each protein sample ID field;
a plurality of crystallization screen ID fields associated with each sample ID;
at least one reagent field(s) associated with each crystallization screen ID field; and
a plurality of crystallization experiment ID fields associated with each crystallization screen ID.
